(19) United States
(12) Patent Application Publication
(10) Pub. No.: US 2026/0053718 A1
(43) Pub. Date: Feb. 26, 2026

(54) POWDER DETERGENT

(71) Applicant: Chia-Wei TENG, Taipei City (TW)

(72) Inventor: Chia-Wei TENG, Taipei City (TW)

(21) Appl. No.: 19/303,805

(22) Filed: Aug. 19, 2025

(30) Foreign Application Priority Data
Aug. 20, 2024 (TW) 113131236

(51) Int. Cl. A61K 8/02; A61Q 19/10

(52) U.S. Cl. CPC A61K 8/022; A61Q 19/10; A61K 2800/51; A61K 2800/52

(57) ABSTRACT

A powder detergent includes sodium coco sulfate, cyclodextrin, jojoba wax ester, a preservative, a chelating agent, and an anticaking agent. Based on a total weight of the powder detergent, the sodium coco sulfate is in an amount ranging from 65 wt % to 85 wt %, the cyclodextrin is in an amount ranging from 3 wt % to 10 wt %, and the jojoba wax ester is in an amount ranging from 1 wt % to 10 wt %.

### CROSS-REFERENCE TO RELATED APPLICATION

This application claims priority to Taiwanese Invention Patent Application No. 113131236, filed on Aug. 20, 2024, the entire disclosure of which is incorporated by reference herein.

### FIELD

The disclosure relates to a detergent, and more particularly to a powder detergent.

### BACKGROUND

Compared to a liquid detergent, a solid detergent not only lowers transportation costs and requires less storage space, but also has a longer shelf life since it contains no water and is thus less likely to breed bacteria and mold. In use, a powder detergent, e.g., a powder dishwashing detergent, only needs to be mixed with water. However, commercially available powder detergents often have disadvantages, such as caking and undesirable dissolution rates in water. Therefore, the commercially available powder detergents are still not the preferred choice for customers.

### SUMMARY

Therefore, an object of the disclosure is to provide a powder detergent that can alleviate at least one of the drawbacks of the prior art.

According to the disclosure, a powder detergent includes sodium coco sulfate, cyclodextrin, jojoba wax ester, a preservative, a chelating agent, and an anticaking agent. Based on a total weight of the powder detergent, the sodium coco sulfate is in an amount ranging from 65 wt % to 85 wt %, the cyclodextrin is in an amount ranging from 3 wt % to 10 wt %, and the jojoba wax ester is in an amount ranging from 1 wt % to 10 wt %.

### DETAILED DESCRIPTION

An embodiment of a powder detergent according to the present disclosure includes sodium coco sulfate, cyclodextrin, jojoba wax ester, a preservative, a chelating agent, and an anticaking agent. Based on a total weight of the powder detergent, the sodium coco sulfate is in an amount ranging from 65 wt % to 85 wt %, the cyclodextrin is in an amount ranging from 3 wt % to 10 wt %, and the jojoba wax ester is in an amount ranging from 1 wt % to 10 wt %.

In the powder detergent of the present disclosure, the sodium coco sulfate serves as a surfactant, the cyclodextrin serves as an anti-odor agent and a stabilizer, and the jojoba wax ester serves as a softener and a lubricant. In certain embodiments, the jojoba wax ester includes jojoba wax PEG-120 esters. In some embodiments, the jojoba wax ester is jojoba wax PEG-120 esters.

In some embodiments, the sodium coco sulfate of the powder detergent in the amount ranging from 65 wt % to 85 wt % allows the powder detergent to form rich foam upon contact with water. The cyclodextrin of the powder detergent in the amount ranging from 3 wt % to 10 wt % may neutralize odor from other ingredients and allow the powder detergent to have good stability. The jojoba wax ester in the amount ranging from 1 wt % to 10 wt % allows the powder detergent to be moisturizing on the skin.

In some embodiments, based on the total weight of the powder detergent, the sodium coco sulfate, the cyclodextrin and the jojoba wax ester are in a total amount of 91 wt %. In some embodiments, based on the total weight of the powder detergent, the sodium coco sulfate is in the amount of 80 wt %, the cyclodextrin is in the amount of 8 wt %, and the jojoba wax ester is in the amount of 3 wt %. The powder detergent has advantages, e.g., a relatively fast dissolution rate, rich foam and superior moisturizing properties.

In some embodiments, based on the total weight of the powder detergent, the preservative is in an amount ranging from 8 wt % to 15 wt %. In some embodiments, based on the total weight of the powder detergent, the preservative is in the amount of 8 wt %. In some embodiments, the preservative includes sodium benzoate. In certain embodiments, the preservative is sodium benzoate.

In some embodiments, based on the total weight of the powder detergent, the chelating agent is in an amount ranging from 0.4 wt % to 2 wt %. In some embodiments, based on the total weight of the powder detergent, the chelating agent is in the amount of 0.5 wt %. In some embodiments, the chelating agent includes tetrasodium glutamate diacetate. In certain embodiments, the chelating agent is tetrasodium glutamate diacetate.

In some embodiments, based on the total weight of the powder detergent, the anticaking agent is in an amount ranging from 0.3 wt % to 1 wt %. In some embodiments, based on the total weight of the powder detergent, the anticaking agent is in the amount of 0.5 wt %. In some embodiments, the anticaking agent includes sodium p-toluenesulfonate. The sodium p-toluenesulfonate may improve wettability, solubility and dispersibility of the powder detergent in water. In certain embodiments, the anticaking agent is sodium p-toluenesulfonate.

In some embodiments, based on the total weight of the powder detergent, the sodium coco sulfate is in the amount of 80 wt %, the cyclodextrin is in the amount of 8 wt %, the jojoba wax ester (e.g., jojoba wax PEG-120 esters) is in the amount of 3 wt %., the preservative is in the amount of 8 wt %, the chelating agent is in the amount of 0.5 wt %, and the anticaking agent is in the amount of 0.5 wt %.

The powder detergent may be used for cleaning skin, such as hands, but is not limited thereto. In addition, the powder detergent is easy to use and convenient to users because the users simply need to dissolve an appropriate amount of the powder detergent in water.

The present disclosure will be further described with reference to the following examples. However, it should be understood that the following examples are merely for illustration purposes and should not be considered as a limitation to the implementing of the present disclosure.

### Example 1

In Example 1, 80 wt % of sodium coco sulfate (commercially available from Stepan Company), 8 wt % of cyclodextrin (commercially available from Baolingbao Biology Co., Ltd.), 3 wt % of jojoba wax PEG-120 esters (commercially available from Hallstar Company), 8 wt % of sodium benzoate, 0.5 wt % of tetrasodium glutamate diacetate, and 0.5 wt % of sodium p-toluenesulfonate were homogeneously mixed to obtain a powder detergent.

### Comparative Example 1

A powder detergent of Comparative Example 1 is a commercial powder detergent (product name: Hand Wash Refill) available from Forgo.

### Comparative Example 2

A powder detergent of Comparative Example 2 is a commercial powder detergent (product name: Hand Soap Refill) available from Blueland.

### Evaluation of Dissolution Rates

Firstly, 10 g of the powder detergent of Example 1 was added into 300 mL of water. An amount of time that it took for the powder detergent to completely dissolve in the water was recorded. A dissolution rate of the powder detergent was calculated according to a formula: an amount of the powder detergent/the amount of time that it took for the powder detergent to completely dissolve in the water. The dissolution rates of Comparative Example 1 and Comparative Example 2 were also respectively estimated according to the aforesaid procedure and formula. The results are presented in Table 1.

TABLE 1

Powder detergent

| | | Comparative | Comparative |
|---|---|---|---|
| | Example 1 | Example 1 | Example 2 |
| The amount of | 10 | 10 | 10 |
| the powder | | | |
| detergent (g) | | | |
| The amount of | 0.25 | 1 | 0.5 |
| time for the | | | |
| powder | | | |
| detergent to | | | |
| completely | | | |
| dissolve (hour) | | | |
| The dissolution | 40 | 10 | 20 |
| rate of the | | | |
| powder | | | |
| detergent | | | |
| (g/hour) | | | |

Referring to Table 1, the powder detergent of Example 1 exhibits a faster dissolution rate than those of Comparative Example 1 and Comparative Example 2. The results reveal that the powder detergent of the Example 1 may dissolve in the water more quickly than those of Comparative Example 1 and Comparative Example 2.

In summary, with the combination of the sodium coco sulfate, the cyclodextrin, the jojoba wax ester, the preservative, the chelating agent, and the anticaking agent, and with the specific weight percentages of the sodium coco sulfate, the cyclodextrin, and the jojoba wax ester, the embodiment of the powder detergent of this disclosure may be quickly dissolved in water without caking, and the detergent may form rich foam upon contact with water. In addition, the powder detergent may not only be free of pungent odor but also have a long shelf life since the powder detergent has resistance against degradation and is less likely to breed bacteria, yeast and mold. When the powder detergent is used for cleaning skin, oil and dirt on the skin may be removed effectively and mildly. Furthermore, the skin may maintain moisture and smoothness after cleaning. Therefore, users may have a pleasant cleaning experience.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A powder detergent comprising:
sodium coco sulfate;
cyclodextrin;
jojoba wax ester;
a preservative;
a chelating agent; and
an anticaking agent,
wherein, based on a total weight of said powder detergent, the sodium coco sulfate is in an amount ranging from 65 wt % to 85 wt %, the cyclodextrin is in an amount ranging from 3 wt % to 10 wt %, and the jojoba wax ester is in an amount ranging from 1 wt % to 10 wt %.

2. The powder detergent as claimed in claim 1, wherein the anticaking agent includes sodium p-toluenesulfonate.

3. The powder detergent as claimed in claim 2, wherein, based on the total weight of said powder detergent, the anticaking agent is in an amount ranging from 0.3 wt % to 1 wt %.

4. The powder detergent as claimed in claim 1, wherein, based on the total weight of said powder detergent, the anticaking agent is in an amount ranging from 0.3 wt % to 1 wt %.

5. The powder detergent as claimed in claim 1, wherein the chelating agent includes tetrasodium glutamate diacetate.

6. The powder detergent as claimed in claim 5, wherein, based on the total weight of said powder detergent, the chelating agent is in an amount ranging from 0.4 wt % to 2 wt %.

7. The powder detergent as claimed in claim 1, wherein, based on the total weight of said powder detergent, the chelating agent is in an amount ranging from 0.4 wt % to 2 wt %.

8. The powder detergent as claimed in claim 1, wherein the preservative includes sodium benzoate.

9. The powder detergent as claimed in claim 8, wherein, based on the total weight of said powder detergent, the preservative is in an amount ranging from 8 wt % to 15 wt %.

10. The powder detergent as claimed in claim 1, wherein, based on the total weight of said powder detergent, the preservative is in an amount ranging from 8 wt % to 15 wt %.

11. The powder detergent as claimed in claim 1, wherein, based on the total weight of said powder detergent, the sodium coco sulfate, the cyclodextrin, and the jojoba wax ester are in a total amount of 91 wt %.

12. The powder detergent as claimed in claim 11, wherein, based on the total weight of said powder detergent, the sodium coco sulfate is in the amount of 80 wt %, the cyclodextrin is in the amount of 8 wt %, and the jojoba wax ester is in the amount of 3 wt %.

13. The powder detergent as claimed in claim 1, wherein, based on the total weight of said powder detergent, the sodium coco sulfate is in the amount of 80 wt %, the cyclodextrin is in the amount of 8 wt %, the jojoba wax ester is in the amount of 3 wt %, the preservative is in an amount of 8 wt %, the chelating agent is in an amount of 0.5 wt %, and the anticaking agent is in an amount of 0.5 wt %.

14. The powder detergent as claimed in claim 13, wherein the jojoba wax ester is jojoba wax PEG-120 esters.

15. The powder detergent as claimed in claim 1, wherein, the jojoba wax ester is jojoba wax PEG-120 esters.
